(19) United States
(12) Patent Application Publication
(10) Pub. No.: US 2025/0186810 A1
(43) Pub. Date: Jun. 12, 2025

(54) METHODS AND APPARATUSES FOR ANESTHETIZING TARGET TISSUE

(71) Applicant: Otsuka Medical Devices Co., Ltd., Tokyo (JP)

(72) Inventors: Dimitri Augustin, San Mateo, CA (US); Liang Zhai, Belmont, CA (US)

(21) Appl. No.: 18/979,511

(22) Filed: Dec. 12, 2024

Related U.S. Application Data
(60) Provisional application No. 63/609,279, filed on Dec. 12, 2023.

(51) Int. Cl. A61N 7/02; A61N 7/00

(52) U.S. Cl. CPC A61N 7/02; A61N 2007/003

(57) ABSTRACT

A system for treating at least one nerve in target tissue in proximity to an AV fistula of a patient having an epidermis may include a ablation element configured to emit ablation energy to the at least one nerve in proximity to an AV fistula; a generator electrically coupled to the transducer, wherein the generator transmits electrical energy to the transducer; and a spacer connected to the ablation element, wherein at least part of the spacer is configured to contact the surface of the epidermis in proximity to the nerves surrounding the AV fistula, and thereby space the ablation element a particular distance from the surface of the epidermis; where the ablation energy is sufficient to anesthetize at least one of the nerves in the target tissue.

This application claims priority to and the benefit of U.S. Provisional Patent Application No. 63/609,279, filed on Dec. 12, 2023, pending, the entire disclosure of the above application is expressly incorporated by reference herein.

### BACKGROUND

High blood pressure, also known as hypertension, commonly affects adults. Left untreated, hypertension can result in renal disease, arrhythmias, and heart failure. Treatment of hypertension has focused on interventional approaches to inactivate the renal nerves surrounding a renal artery. Chronic kidney disease affects more than 1 in 7 adults in the United States. Early stages of kidney disease can be treated by exercise, medications, lifestyle changes, and controlling the patient's diabetes and hypertension. However, end stage kidney disease requires dialysis and, in more serious cases, kidney transplant. Over a half million Americans receive dialysis in any given year, with over one hundred thousand new diagnoses annually. Japan has the second largest number of patients receiving chronic maintenance dialysis worldwide, after the United States. The number of chronic dialysis patients in Japan has been increasing annually, and exceeded 340,000 in 2019.

Dialysis patients receive treatment 3-4 times a week, where a dialysis machine takes over the function of the kidneys and filters out toxins in a patient's blood. An arteriovenous (AV) fistula is often created in a patient to provide a long-lasting access point for dialysis. Creating an AV fistula involves a surgical procedure that connects an artery to a vein, e.g., the radial artery may be connected surgically to the cephalic vein in a patient's wrist to create a radial cephalic fistula. The AV fistula provides a high flow channel that is suitable for dialysis. About three months after surgery to form the AV fistula, needles may be inserted into the AV fistula. AV fistula access requires the use of two needles inserted into the AV fistula, spaced apart from one another, for each hemodialysis session: one on the blood removal side and one on the blood return side. AV fistula accounts for about 60% of vascular access in dialysis in the United States and 96.8% in Japan.

### SUMMARY

An exemplary system for treating at least one nerve in target tissue in proximity to an AV fistula of a patient having an epidermis may include a ablation element configured to emit ablation energy to the at least one nerve in proximity to an AV fistula; a generator electrically coupled to the transducer, wherein the generator transmits electrical energy to the transducer; and a spacer connected to the ablation element, wherein at least part of the spacer is configured to contact the surface of the epidermis in proximity to the nerves surrounding the AV fistula, and thereby space the ablation element a particular distance from the surface of the epidermis; where the ablation energy is sufficient to anesthetize at least one of the nerves in the target tissue.

An exemplary method for treating at least one nerve in target tissue in proximity to an AV fistula of a patient may include placing an ablation element associated with a spacer in proximity to the target tissue; cooling tissue in proximity to the at least one nerve; and energizing the ablation element to deliver ablation energy to the target tissue until at least one nerve in the target tissue is anesthetized.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “Figure” and “FIG.” herein), of which:

FIG. 1A shows a perspective section view of layers of the human skin.

FIG. 1B shows a schematic of an exemplary system for anesthetizing target tissue or anesthetizing system according to many embodiments. The anesthetizing system may include a catheter or carrier having a proximal end and a distal end.

FIGS. 1C-1D show section views of an exemplary anesthetizing system in use, the system including an ablation element configured to deliver ablation energy to target tissue of a subject near a blood vessel or an arteriovenous fistula, and a covering configured to position the ablation element relative to a portion of skin at a position at or near the target tissue.

FIGS. 1E-1F show section views of an exemplary anesthetizing system in use, the system including a carrier configured to be placed through at least one bodily vessel of a subject, and a covering configured to position the ablation element relative to a bodily vessel at or near the target tissue.

FIG. 2A shows a side section view of an exemplary anesthetizing system including an ablation element coupled at a distal end of a carrier.

FIG. 2B shows a side section view of an exemplary anesthetizing system including an ablation element positioned on a distal end of a carrier.

FIG. 2C shows a side section view of an exemplary anesthetizing system including an ablation element positioned along a carrier.

FIG. 3A shows a bottom view of a covering including a retention layer and a border configured to secure the covering to the portion of skin according to an example.

FIG. 3B shows a bottom view of a covering including an insolation layer configured to retain at least a portion of the ablation energy according to an example.

FIG. 3C shows a bottom view of a covering including at least one port configured to aid in filling an enclosed area formed by securing the covering to the portion of skin according to an example.

FIG. 4A shows a side section view of an exemplary anesthetizing system including at least one spacer configured to position the system relative to a portion of skin. FIG. 4A shows the portion of the skin at the position at or near the target tissue, which includes at least a portion of the dermis or at least a portion of the epidermis.

FIG. 4B shows a side section view of an exemplary anesthetizing system including at least one adjustable spacer configured to position the system relative to a portion of skin.

FIG. 4C shows a side section view of an exemplary anesthetizing system including at least one spacer and at least one adjustable spacer configured to position the system relative to a portion of skin.

FIG. 5A shows a side view of an exemplary dome spacer configured to maintain a fixed distance between the system and the portion of skin.

FIG. 5B shows a side view of an exemplary adjustable dome spacer configured to adjust a distance between the system and the portion of skin.

FIG. 5C shows a side section view of an exemplary anesthetizing system including a dome spacer having a cover configured to maintain a transducing medium between the system and the portion of skin.

FIG. 6A shows a side view of a ablation element configured to deliver energy to the target tissue in a substantially unidirectional direction according to an example.

FIG. 6B shows a side view of a ablation element including a series of focal points configured to deliver focused energy to the target tissue in a substantially focused direction according to an example.

FIG. 6C shows a side view of a ablation element including a cylindrical transducer configured to deliver diffuse or unfocused energy.

FIG. 6D shows a side view of a ablation element including a deflective covering configured to one or more of modify, deflect, or redirect at least a portion of the ablation energy generated by the ablation element.

FIG. 6E shows a side view of a deflective covering including at least one layer of a deflective or dampening material according to an example.

FIG. 6F shows a side view of a deflective covering including a sensor according to an example.

FIG. 6G shows a perspective view of a ablation element with a single transducer element configured to deliver energy to a focal point according to an example.

FIG. 6H shows a perspective view of a ablation element with multiple transducer elements configured to deliver energy to a focal point according to an example.

FIG. 6I shows a perspective view of a ablation element with a single transducer element configured to deliver energy to a focal point and a target volume according to an example.

FIG. 6J shows a perspective view of a ablation element with multiple transducer elements configured to deliver energy to a focal point and a target volume according to an example.

FIG. 6K shows a section view of a cylindrical ablation element configured to deliver energy to a focal line and target volume according to an example.

FIG. 6L shows a section view of a ablation element with a waveguide according to an example.

FIG. 6M shows a section view of a ablation element with a lens according to an example.

FIGS. 7-8 are flow charts describing exemplary methods for anesthetizing target tissue at or adjacent a portion of skin utilizing exemplary systems described herein.

### DETAILED DESCRIPTION

While various embodiments of the present disclosure have been shown and described herein, it will be obvious to those skilled in the art that such embodiments are provided by way of example only. Numerous variations, changes, and substitutions may occur to those skilled in the art without departing from the present disclosure. It should be understood that various alternatives to the embodiments of the present disclosure described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The terms “about,” “substantially,” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

The present disclosure relates generally to minimally invasive apparatuses, systems, and methods that provide energy delivery to a targeted anatomical location of a subject to decrease the pain and/or the sensation of pain experienced by a subject at the needle insertion site prior to and/or during dialysis, while also decreasing the length of time that must be spent on prep. Both topically applied (extracorporeal) and catheter-based, intraluminal (intravascular) devices and systems are disclosed, which are configured to deliver ultrasonic energy to treat tissue, such as nerve tissue.

Currently, antiseptic numbing cream, lidocaine tape, icepacks, and guided needles are used to reduce the pain and/or reduce the sensation of pain experienced by a subject at the needle insertion site (i.e., puncture site) at an AV fistula for dialysis. Numbing time increases the time required for the procedure (three to five hours) by thirty to sixty minutes. Insertion of the needles can still be quite painful and given that dialysis must be performed 3-4 times a week, presents a terrible burden and source of stress to the patient.

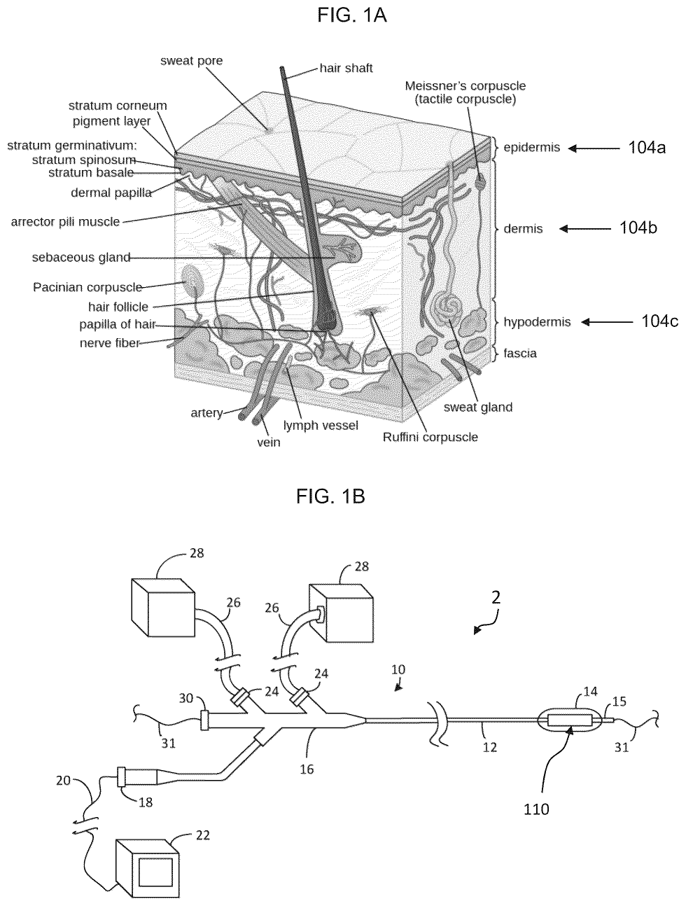

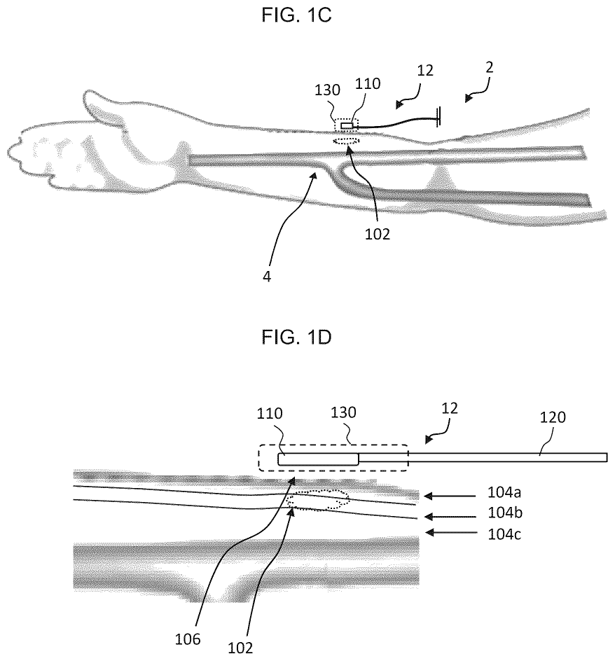

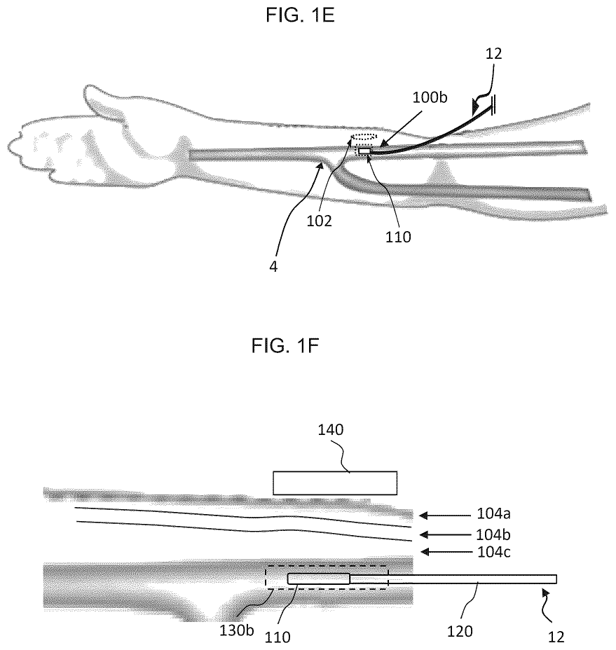

Referring to FIG. 1A, the skin is primarily made up of three layers: the epidermis layer (EL) 104a which is generally up to 1.5 mm thick and comprises free nerves or nociceptors that respond to pain, light touch, and temperature variations, the dermis layer (DL) 104b which is about 0.6 mm to 4 mm thick and comprises a reticular layer with a rich sensory and sympathetic nerve supply and a papillary dermis, and the subcutaneous tissue layer (SL) 104c which includes fat and connective tissue. The subcutaneous tissue layer may be referred to as the hypodermis; the terms are synonymous. The nerve cell endings that initiate the sensation of pain are referred to as nociceptors. While nociceptors may be present in each of the three layers of the skin, most nociceptors typically are located in the dermis layer.

Where an AV fistula is created for dialysis, that AV fistula is almost always in an arm of a patient. That AV fistula typically is not far beneath the skin, which tends to be thin on the volar forearm: the portion of the forearm that is on the same side as the palm of the hand.

### System

Referring to FIG. 1B, an exemplary anesthetizing system 2 is shown. The anesthetizing system 2 may include a catheter 10 having a proximal end and a distal end. The catheter 10 may include a shaft 12, a spacer 130, and a tip member 15. The spacer 130 can be positioned between the catheter shaft 12 and the tip member 15. The spacer 130 can be or include a compliant, semi-compliant, or non-compliant medical balloon. Suitable materials for the spacer 130 may include, but are not limited to nylon, polyimide films, thermoplastic elastomers such as those marked under the trademark PEBAX™, medical-grade thermoplastic polyurethane elastomers such as those marketed under the trademarks PELLETHANE® or ISOTHANE®, and other suitable polymers or any combination thereof.

The catheter 10 can have a hub 16 at the proximal end of the catheter shaft 12. The hub 16 can include one or more electrical couplings 18 for connecting the catheter system to one or more external electrical conductors 20 that are each in electrical communication with electronics 22, e.g., a generator. Suitable external electrical conductors 20 include, but are not limited to, wires, cables, and Flexible Printed Circuits (FPC).

The electronics 22 can control the catheter 10 to sweep the operating frequency and can control the durations of the individual pulses and total time of series of pulses to control the temperature in the target tissue and create a lesion therein.

The catheter shaft 12 can include one or more electrical lumens. Each of the electrical lumens may extend from one or more of the electrical couplings 18 along a longitudinal length of the catheter shaft 12 toward a distal end of the catheter shaft 12. The electrical lumens can each hold one or more electrical conductor carriers that each carry one or more electrical conductors. The electrical conductors can be in electrical communication with the electronics 22 through the electrical coupling 18 and one or more of the external electrical conductors 20. Suitable electrical conductors include, but are not limited to, wires, insulated wires, cables, and Flexible Printed Circuits (FPC). When an electrical conductor carrier carries multiple electrical conductors, a suitable electrical conductor carrier can be an electrically insulating jacket. When an electrical conductor carrier carries a single electrical conductor, an electrical insulator on the electrical conductor can serve as the electrical conductor carrier.

The hub 16 can include one or more fluid ports 24 for connecting the catheter to one or more conduits 26. Suitable conduits 26 include, but are not limited to, tubes and hoses. One or more conduits may conduit 26 can provide fluid communication between each fluid port 24 and a fluid source/fluid collector 28. Suitable fluid sources 28 include, but are not limited to, cartridges, pumps, tanks, reservoirs, and vessels. The catheter shaft 12 can include one or more fluid lumens. Each of the fluid lumens can be in fluid communication with one of the fluid ports 24 along a longitudinal length of the catheter shaft 12 toward a distal end of the catheter shaft 12.

The hub 16 can include one or more guidewire ports 30 for receiving a guidewire 31. The catheter shaft 12 can include a guidewire lumen. The guidewire lumen can extend along a longitudinal length of the catheter shaft 12 toward a distal end of the catheter shaft 12. The guidewire lumen can be in fluid communication with the guidewire port 30 such that a guidewire 31 inserted into the guidewire port 30 can be received within the guidewire lumen.

Turning to FIGS. 1C-1D, an exemplary anesthetizing system 2 can include a ablation element 110 coupled to a shaft 12 configured to deliver ablation energy, e.g., ultrasonic acoustic energy, RF energy, microwave energy, and/or other suitable energy to target tissue 102 of a subject, and a spacer 130 configured to position the ablation element 110 a suitable distance from the surface of the epidermis 104a, at a position at or near the target tissue 102.

An AV fistula 4 is generally created in proximity to the surface of the volar forearm, which is the portion of the forearm that is on the same side as the palm of the hand. The mean epidermal thickness of the volar forearm has been reported to be 75.5 μm, with a lower bound of 61.7 μm and an upper bound of 89.3 μm. (Lintzeri et. al., (2022), Epidermal thickness in healthy humans: a systematic review and meta-analysis. J Eur Acad Dermatol Venereol, 36:1191-1200 (2022); https://doi.org/10.1111/jdv.18123). When a clinician creates an AV fistula 4 in a patient, the depth of that AV fistula 4 below the surface of the skin of the volar forearm is 0.6 cm or less, to allow successful dialysis. (Banerjee, S., Fistula maturation and patency for successful dialysis; Dial. Transplant., 38:442-442 (2009); https://doi.org/10.1002/dat.20369). Thus, when a clinician inserts a needle into the AV fistula 4 for dialysis, that needle generally passes through up to 0.6 cm of skin and tissue to enter the AV fistula 4. The length of the AV fistula 4 is generally 5 to 6 cm. (Banerjee, S., Fistula maturation and patency for successful dialysis; Dial. Transplant., 38:442-442 (2009); https://doi.org/10.1002/dat.20369). Thus, the target tissue 102 for anesthetization occupies is located between the AV fistula 4 and the surface of the epidermis 104a. Anesthetizing that target tissue 102 eliminates, or significantly reduces, pain caused by the insertion of one or two dialysis needles through the target tissue 102 into the AV fistula 4. Pain caused by insertion of the dialysis needle or needles into the AV fistula 4 is not just from the pain of the needle stick itself, but also from the relatively large size of the dialysis needle or needles. A dialysis needle is generally 14 to 18 gauge (a 14 gauge needle is 2.1 mm in outer diameter) in order to match the high blood flow rate at the AV fistula 4. Typically, the larger the needle gauge, the more pain a patient experiences upon insertion of that needle, but the higher the blood flow therethrough.

In some embodiments, the target tissue 102 is substantially 5-10 cm long (along the volar forearm, for example), substantially 1 cm wide, and substantially 1-5 mm deep, where depth is measured substantially perpendicular to the epidermis, and where measurement of that depth begins 0.5-1 mm below the surface of the epidermis 104a, with a corresponding volume of substantially 1-5 cm³. The depth of the target tissue 102 begins at 0.5-1 mm below the surface of the epidermis 104a in order to prevent damage to the surface of the epidermis 104a. Such damage would interfere with successful dialysis, because the dialysis needles could not be inserted through that damaged tissue. For patients with thin, friable skin, which include many elderly patients, the depth of the target tissue 102 advantageously may begin closer to 1 mm below the surface of the epidermis 104a to ensure that the surface of the epidermis 104a is not damaged. The length, width, depth, and volume of target tissue 102 in a particular patient depends on the anatomy of that patient and the particular location and dimensions of the AV fistula 4 created in that patient. The largest variable between patients is the depth of the AV fistula 4, and that depth is typically 1-5 mm. While the AV fistula 4 may be deeper than 5 mm, those AV fistulas 4 are rare, because one reason to create the AV fistula 4 is to create a space close to the surface of the epidermis 104a to make it easier to insert dialysis needles thereinto. Due to the size of the AV fistula 4 and the fact that the two dialysis needles are spaced apart from one another, the target tissue 102 may be in two sections spaced apart from one another. In such embodiments, the target tissue 102 still may have a volume of substantially 1-5 cm³, split between two separate sections spaced apart from one another. Each section may be substantially 2.5-5 cm long, substantially 1 cm wide, and substantially 1-5 mm deep. The depth of the target tissue 102 may extend into the AV fistula 4 itself a small amount without ill effects, because a mature AV fistula 4 is typically at least 6 mm in diameter. However, it is undesirable that the target tissue 102 extends all the way to the back of the AV fistula 4.

The target tissue 102 may be near a blood vessel or an AV fistula 4, and the portion of the skin at the position at or near the target tissue can include one or more of at least a portion of the dermis 104b or at least a portion of the epidermis 104a. In some embodiments, the target tissue 102 includes a portion of the dermis 104b but not a portion of the epidermis 104a. In certain embodiments, the spacer 130 may be configured to one or more of modify or redirect at least a portion of the ablation energy from a direction orthogonal to the target tissue and direct a modified energy towards the target tissue. For example, a modified energy can include superposition of two or more energy signals, such as where energy is additive through constructive interference and subtractive through destructive interference. In certain embodiments, the spacer 130 can be configured to position the ablation element 110 relative to one or more of at least a portion of the epidermis 104a or at least a portion of the dermis 104b.

The ablation element 110 may include at least one of an ultrasound transducer, a radiofrequency source, a pulsed field element, a cryoablation element, or a chemical-ablation element. In the embodiments described herein, the ablation element 110 is one or more ultrasonic transducers, which utilize acoustic energy for neuromodulation. The ablation element 110 is configured to emit ablation energy to anesthetize at least a portion of the target tissue 102 without substantially injuring the skin or other tissue, such as intervening tissue (tissue between the ablation element 110 and the target tissue 102) or tissue unintended to be ablated that is adjacent the target tissue. In an example, the ablation element 110 is configured to emit ablation energy sufficient to denervate some or all of the target tissue.

In certain embodiments, the ablation element 110 is configured to emit ablation energy which heats the target tissue to temperatures below 50° C. (from between ambient temperature and body temperature) for up to 7 seconds to cause anesthetization. In other embodiments, the ablation element 110 is configured to emit ablation energy which heats the target tissue to a temperature or temperatures higher than 50° C. or for more than 7 seconds to cause anesthetization. The ablation element 110 may be configured to emit ablation energy to cause modification, deactivation, disablement, denervation, damage, electroporation, apoptosis, necrosis, thermal alteration and destruction, acoustic heating, and/or injury of the target tissue 102.

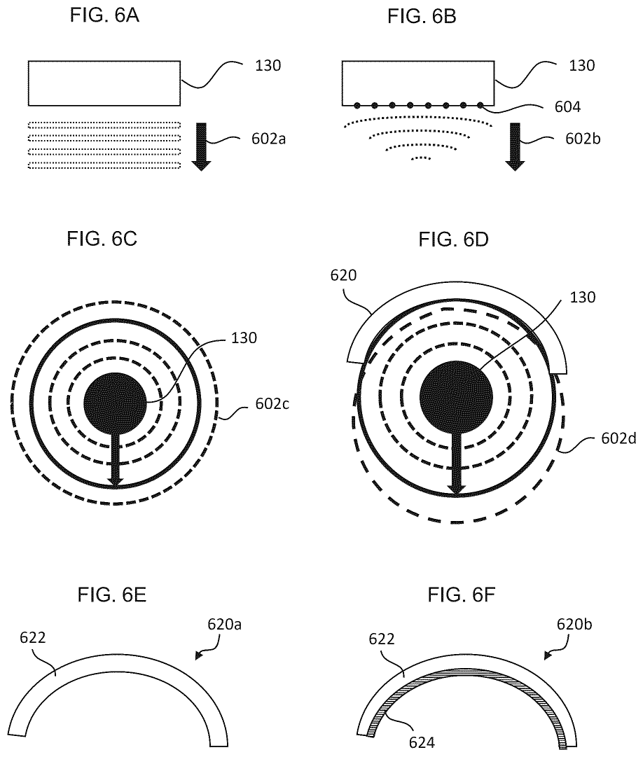

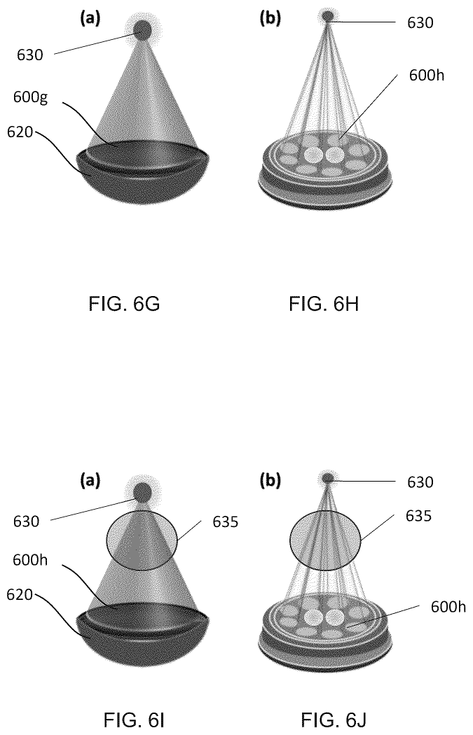

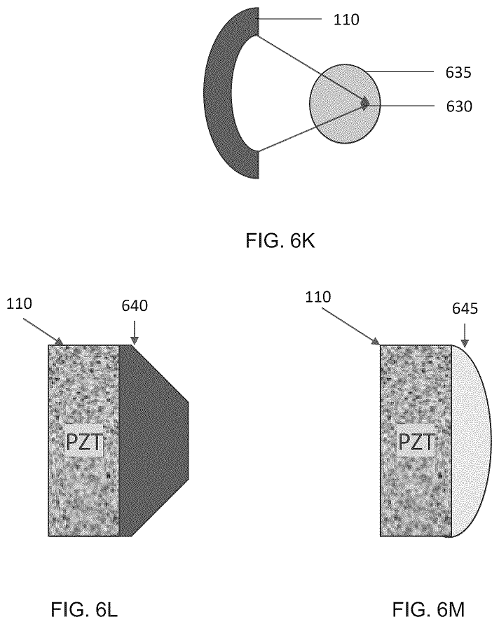

In certain embodiments, the ablation element 110 may be shaped to direct the ablation energy to the target tissue 102. In other words, rather than emitting energy omnidirectionally and radially outward, as a cylindrical transducer would, the ablation element 110 may be configured to emit energy substantially unidirectionally toward the target tissue 102. To accomplish this, the ablation element 110 may be substantially flat (FIG. 6A) or slightly curved, such as in embodiments in which the ablation element 110 is an ultrasonic transducer, the ablation element 110 delivers energy to the target tissue 102 generally linearly along a direction 602a. The ablation element 110 may have other suitable shapes, such as in embodiments in which the ablation element 110 delivers energy other than ultrasonic energy.

In some embodiments, the ablation element 110 may have substantially the same thickness along its length. In other embodiments, the ablation element 110 may vary in thickness at one or more locations along its length. Where the ablation element 110 is an ultrasonic transducer, varying the thickness along the ablation element 110 may cause the ultrasonic transducer to emit acoustic energy outward in a controlled direction or directions to facilitate treatment of the target tissue 102. In some embodiments, in which the ablation element 110 is an ultrasonic transducer, different thicknesses of the ablation element 110 at different locations thereon may be utilized to generate different acoustic frequencies. As a result, a portion of the ablation element 110 that emits ultrasound at a lower frequency may penetrate more deeply into tissue, and a portion of the ablation element 110 that emits ultrasound at a higher frequency may penetrate more shallowly into tissue. In some embodiments, the ablation element 110 may be an array of smaller elements, such as ultrasound transducers, that may be controlled to direct or shape the overall emission of ultrasound therefrom in order to tune or shape the acoustic energy emitted from the ablation element 110 as a whole.

Parameters of ultrasound energy emitted from the ablation element 110, when the ablation element 110 is an ultrasound transducer, may include an intensity, a frequency, an amplitude, a pulse width, a phase, a duration, a modality, a temperature, and an orientation relative to a spatial dimension. In an example, the ablation element 110 that is an ultrasound transducer is configured to emit ultrasound energy at a frequency of substantially 9 MHz. In other examples, the ablation element 110 that is an ultrasound transducer is configured to emit ultrasound energy at a frequency in the range of 5-30 MHZ, in the range of 5-25 MHZ, in the range of 5-20 MHZ, in the range of 8-25 MHz, in the range of 8-20 MHz, in the range of 8-15 MHZ, in the range of 8-12 MHZ, or in the range of 8-10 MHz. In one example, the ablation element 110 may be configured to generate ultrasound energy that causes high temperatures in target tissue located within the dermis, at a distance spaced apart from the ablation element 110. The amount of energy, and its characteristics, are generated and delivered by the ablation element 110, and depend on factors such as the design of the ablation element 110, the energy supplied to the ablation element 110, the time of treatment, the distance to the target tissue, and the characteristics of intervening tissue adjacent to the target tissue. In some embodiments, the ablation element 110 may be configured to deliver energy at an average surface acoustic intensity of 5 W/cm² or less, 5 W/cm²-15 W/cm², or 15 W/cm²-25 W/cm². In embodiments in which the ablation element 110 is an ultrasonic transducer, the ablation element 110 may be configured to deliver acoustic energy at an entry acoustic intensity between 5 and 25 W/cm².

The acoustic dose of ultrasound energy delivered to target tissue 102 is the power of the ultrasound energy over the duration of time that ultrasound energy is applied. Thus, the duration of delivery of ultrasound energy into target tissue 102 is directly proportional to the average surface acoustic intensity delivered by the ablation element 110 that is an ultrasound transducer. The higher the surface acoustic intensity, the less duration is required for a given acoustic dose. Regardless of the duration and the surface acoustic intensity, in some embodiments the acoustic dose is selected and delivered to cause the target tissue 102 to rise to a target temperature after the acoustic dose has been administered. In some embodiments, the target temperature is a minimum temperature throughout the target tissue 102. In some embodiments, the target temperature is a maximum temperature throughout the target tissue 102. In other embodiments, the target temperature is an average temperature within the target tissue 102. In some examples, the target temperature is substantially 60° C. In other examples, the target temperature is substantially 48° C. In other examples, the target temperature is in the range of 48-60° C., 50-60° C., 52-60° C., 50-58° C., 52-58° C., or 54-56° C. In some examples, the target temperature is over 60° C.

In some examples, the duration of the acoustic dose is substantially 0.1 second, and the average surface acoustic intensity is selected accordingly to produce a temperature of at least about 60° C. in the target tissue 102 by the end of that duration. In other examples, the duration across which ultrasound energy is delivered into the target tissue 102 is 0.05-2 seconds, 0.05-1.5 seconds, 0.05-1 second, 0.05-0.75 seconds, 0.05-0.5 seconds, 0.05-0.4 seconds, 0.05-0.3 seconds, 0.05-0.2 seconds, 0.05-0.1 seconds, 0.08-2 seconds, 0.08-1.5 seconds, 0.08-1 second, 0.08-0.75 seconds, 0.08-0.5 seconds, 0.08-0.4 seconds, 0.08-0.3 seconds, 0.08-0.2 seconds, 0.08-0.1 seconds, 0.1-2 seconds, 0.1-1.5 seconds, 0.1-1 second, 0.1-0.75 seconds, 0.1-0.5 seconds, 0.1-0.4 seconds, 0.1-0.3 seconds, 0.1-0.2 seconds, 0.2-2 seconds, 0.2-1.5 seconds, 0.2-1 second, 0.2-0.75 seconds, 0.2-0.5 seconds, 0.2-0.4 seconds, 0.2-0.3 seconds, 0.5-2 seconds, 0.5-1.5 seconds, 0.5-1 second, or 0.5-0.75 seconds. In other examples, the duration across which ultrasound energy is delivered into the target tissue 102 is 0.1-30 seconds, 0.1-25 seconds, 0.1-20 seconds, 0.1-15 seconds, 0.1-10 seconds, 0.1-5 seconds, 0.5-30 seconds, 0.5-25 seconds, 0.5-20 seconds, 0.5-15 seconds, 0.5-10 seconds, 0.5-5 seconds, 1-30 seconds, 1-25 seconds, 1-20 seconds, 1-15 seconds, 1-10 seconds, 1-5 seconds, 2-30 seconds, 2-25 seconds, 2-20 seconds, 2-15 seconds, 2-10 seconds, 2-5 seconds, 5-30 seconds, 5-25 seconds, 5-20 seconds, 5-15 seconds, 5-10 seconds, 10-30 seconds, 10-25 seconds, 10-20 seconds, or 10-15 seconds.

In some embodiments, the ablation element 110 is an ultrasonic transducer configured to emit unfocused ultrasound energy. As described in US Pat. App. Pub. 2024/0374933, which is hereby incorporated by reference herein in its entirety, exact precision is not required when unfocused ultrasound is utilized for neuromodulation. The spacer 130 is utilized to space the ablation element 110 apart from the epidermis 104a of patient a distance sufficient to deliver unfocused ultrasound most efficiently to the target tissue 102, which is generally located from the surface of the epidermis 104a to the portion of the AV fistula 4 furthest from the surface of the epidermis 104a.

In some embodiments, the ablation element 110 may be an ultrasonic transducer configured to emit focused ultrasonic energy in a substantially focused direction according to an example (FIG. 6B). In an example, the ablation element 110 may be configured to send ultrasonic energy at different phases to the series of focal points 604 to create the focused energy. In an example, the ablation element 110 used to emit focused ultrasound energy may be curved.

A spacer 130 may be connected to or otherwise associated with the ablation element 110. The spacer 130 may provide an appropriate distance between the ablation element 110 and the target tissue 102 such that unfocused acoustic energy, or other form of ablation energy, is absorbed effectively by the target tissue 102, at the distance the target tissue 102 is located from the ablation element 110. In some embodiments, the spacer 130 may insulate or otherwise protect a clinician from ablation energy emitted by the ablation element 110 in use. The spacer 130 may be fabricated from silicone, plastic, polyurethane, or any other suitable material that facilitates delivery of ablation energy from the ablation element 110 to the target tissue 102, while protecting the clinician and offering case of use during operation. In some embodiments, the appropriate distance between the ablation element 110 and the target tissue 102 may be small; for example, that distance may be in the range of 0.1-1 mm. In such embodiments, the spacer 130 may be simply a layer of transducing gel applied to the surface of the epidermis 104a of the patient. That layer of transducing gel both spaces the ablation element 110 apart from the surface of the epidermis 104a and provides acoustic coupling between the ablation element 110 and the surface of the epidermis 104a.

In embodiments in which the ablation element 110 is an ultrasonic transducer, the spacer 130 may facilitate acoustic coupling between the ablation element 110 and the surface of the epidermis 104a above the target tissue 102. Such acoustic coupling is important, because the interface between air and tissue tends to reflect rather than transmit ultrasound energy. The spacer 130 may provide acoustic coupling by holding a transducing medium such as a gel or liquid therein. Such a transducing medium may be applied into the spacer 130 by a clinician prior to placing the spacer 130 onto the skin of a patient. The spacer 130 may be configured to seal against the skin of the patient securely enough to hold the transducing medium therein during treatment, thereby facilitating the transfer of acoustic energy from the ablation element 110 to the target tissue 102. In other embodiments, the spacer 130 may be prefilled with transducing medium for acoustic coupling, and that transducing medium may be held in place with a membrane or other structure. In this way, the spacer 130 is provided to a clinician with an appropriate acoustic coupling substance, such that the anesthetizing system 2 is simpler to use. In other embodiments, the transducing medium may be scaled in a pod having a shape corresponding to the spacer 130, such that the spacer 130 may be sterilized and reused, and a new pod containing transducing medium can be inserted into the spacer 130 before each use. Optionally, the spacer 130 and/or transducing medium may be cooled prior to use to create a cooling effect on the surface of the skin in use. In other embodiments, the spacer 130 may be configured to cool the transducing medium actively during use of the anesthetizing system 2, for greater patient comfort. In other examples, the patient may place their arm in a bath of water or saline solution during the procedure, to provide cooling to the arm and the skin above the target tissue. In other examples, the spacer 130 and the transducing medium may be omitted.

Turning to FIGS. 6C-6F, an exemplary anesthetizing system 2 is shown including a spacer 130 configured to modify, deflect, and/or redirect at least a portion of the ablation energy emitted from the ablation element 110. In an example, the spacer 130 may be configured to modify the ablation energy from a direction orthogonal to one or more of the portion of skin or target tissue and direct the modified energy 602d towards the one or more of the portion of skin or the target tissue 102. In certain embodiments, the spacer 130 can be configured to facilitate direction of at least a portion of the ablation energy toward the target tissue 102. In an example, the spacer 130 may be configured to respond to at least a portion of the ablation energy. In an example, the responding includes at least one of a change of color, opacity, or viscosity. In an example, the spacer 130 includes at least one layer of a deflective or dampening material 622 configured to respond to at least a portion of the ablation energy (FIG. 6E). In an example, the spacer 130 includes a sensor 624 configured to sense ablation energy (FIG. 6F). In an example, the sensor 624 can be a thin-film array sensor or any other sensor.

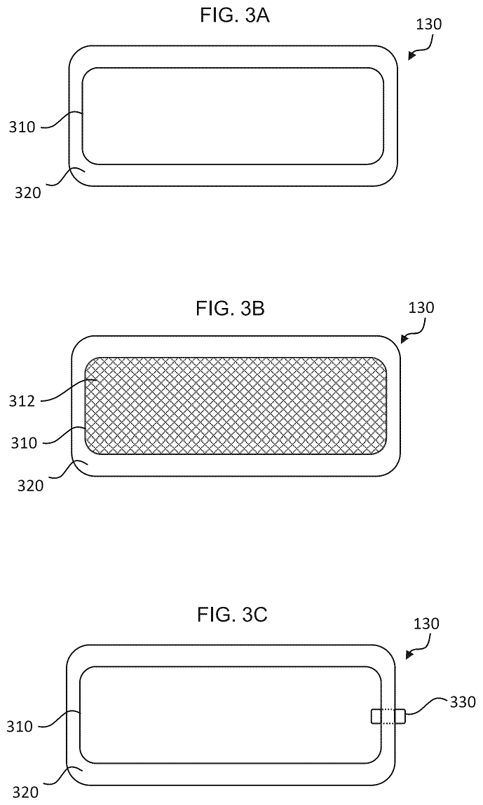

Turning to FIGS. 3A-3C, in certain embodiments, the spacer 130 includes a retention layer 310 configured to retain a transducing medium and/or a border 320 configured to secure the covering to the portion of skin. In some examples, the border 320 can include an adhesive on an underside or can have a lip such that an adhesive can be placed on top of the border. In certain embodiments, a spacer 130 includes an insolation layer 312 configured to retain at least a portion of the ablation energy. In certain embodiments, a spacer 130 includes at least one port 330 configured to aid in filling an enclosed portion formed by securing the covering to the portion of skin according to an example (FIG. 3C). In an example, the spacer 130 can be filled by the one or more fluid ports 24 in communication with the hub 16.

In certain embodiments, an exemplary anesthetizing system 2 includes at least one spacer 130 that includes a fluid port 24 for filling the spacer 130, or at least part of a volume defined by the spacer 130, with a cooling agent and/or transducing medium, in whole or in part. In an example, the spacer 130 can be in communication with the conduit 26 and can provide fluid communication between the fluid port 24, the fluid source 28, and the volume formed by securing the spacer 130 to the upper surface of the epidermis 104a.

The spacer 130 can be at least one of a fixed dimension spacer 420, an adjustable spacer 422, and a dome spacer 500a, 500b, 500c.

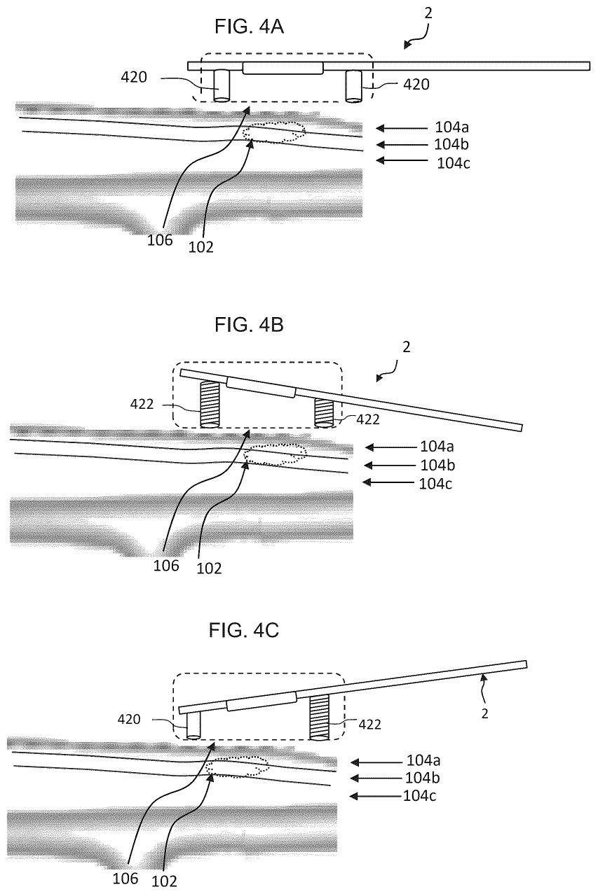

Referring to FIG. 4A, in some embodiments the anesthetizing system 2 can include at least one fixed dimension spacer 420 distal to the ablation element 110 and at least one fixed dimension spacer 420 proximal to the ablation element 110 (FIG. 4A). In an example, the fixed dimension spacer 420 can be fixed in length such that when pressed against the skin, the ablation element 110 will be at a predetermined distance or orientation relative to a surface of the epidermis 104a or the target tissue 102, which may include the epidermis 104a and/or the dermis 104b. Examples of predetermined distances can include any distance between about 0.5 mm to about 6 mm.

In certain embodiments, an anesthetizing system 2 can include at least one adjustable spacer 422 configured to position the ablation element 110 relative to the surface of the epidermis 104a above the target tissue 102. In an example, the adjustable spacer 422 can be adjusted to modify at least one of distance or orientation relative to one or more of a surface of the epidermis 104a or target tissue 102. In an example, the anesthetizing system 2 can include at least one adjustable spacer 422a distal to the ablation element 110 and at least one adjustable spacer 422b proximal to the ablation element 110 (FIG. 4B). In an example, each adjustable spacer 422a, 422b can adjust its length to result in any distance between about 0.5 mm to about 6 mm. In an aspect, the adjustment can result in a change in angle or orientation of the ablation element 110 relative to the target tissue or another tissue. In certain embodiments, an anesthetizing system 2 can include at least one spacer 420 or one adjustable spacer 422a distal to the ablation element 110 and at least one fixed dimension spacer 420 or one adjustable spacer 422 proximal to the ablation element 110 (FIG. 4C).

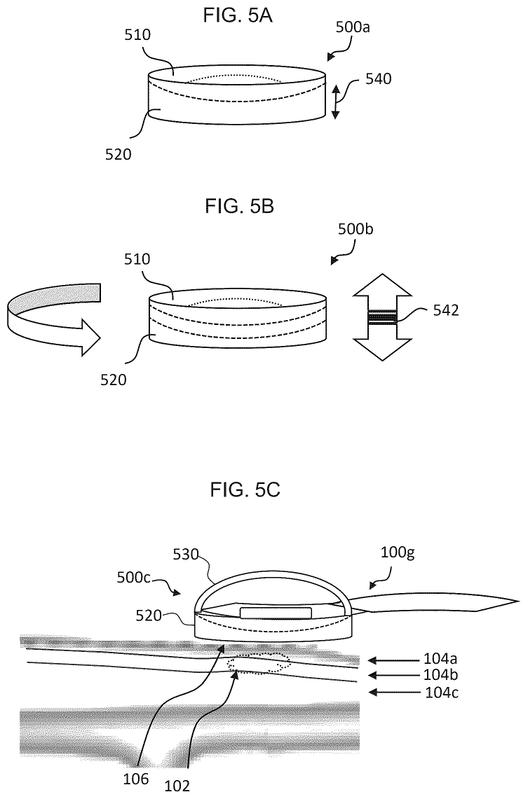

The spacer 130 may have any suitable shape, such as but not limited to a cylinder, a box, or a dome. Turning to FIGS. 5A-5C, in certain embodiments, the spacer 130 may be a dome spacer 500a, 500b, or 500c, having a curved bottom portion 520 configured to position the ablation element 110 relative to the surface of the epidermis 104a above the target tissue 102, and a cover 530 configured to maintain a transducing medium between the ablation element 110 and the surface of the epidermis 104a above the target tissue 102. In an example, a top portion 510 of the spacer 130 can have a dome shape. In an aspect, a shape of a dome spacer 500a, 500b, 500c may be configured to simultaneously space the system from the portion of skin as well as modify an orientation of the ablation element 110 relative to a portion of skin or the target tissue 102, which may include the epidermis 104a and the dermis 104b. In an aspect, the curved bottom portion 520 may be configured to modify an orientation of the ablation element 110 relative to the target tissue 102 and/or the surface of the epidermis 104a when the system may be positioned at different locations within or relative to the curved bottom portion 520.

Referring to FIG. 5A, a dome spacer 500a can be configured to maintain a fixed distance 540 between the ablation element 110 and a surface of the epidermis 104a. Referring to FIG. 5B, an adjustable dome spacer 500b may be configured to adjust an adjustable distance 542 between the ablation element 110 and a surface of the epidermis 104a. In some embodiments, at least one of the top portion 510 and curved bottom portion 520 may be configured to adjust its distance relative to the other. In an example, the top portion 510 and curved bottom portion 520 can have mating threads or are pressure or friction-fitted to adjust their distance relative to the other.

Where the ablation element 110 is configured to emit RF energy, microwave energy, or energy other than ultrasound energy, the spacer 130 may be omitted. Forms of energy other than ultrasound may not travel effectively through air or tissue, and may be more effective when placed in contact with skin or other tissue of the patient, rather than being spaced apart from skin or other tissue.

The embodiments of the ablation element 110 and spacer 130 described above have been described as suitable for placement outside the body of a patient (extracorporcally) in use. To deliver sufficient energy to the target tissue 102, the target tissue 102 may need to be cooled during neuromodulation. The embodiments described above for extracorporeal use during the surgery that creates the AV fistula 4. During that surgery, depending on the patient and the choice of the clinician, the patient is placed under general anesthesia, regional anesthesia, or local anesthesia. In all of those cases of surgical anesthesia, the patient would not experience pain when neuromodulation was applied to target tissue 102 during the surgery performed to create the AV fistula 4. Further, the extracorporeal administration of ablation energy from the ablation element 110 may assist in the anesthesia of the target tissue during the surgery to create the AV fistula 4. Such extracorporeal administration of ablation energy also does not require a separate entry into the body of the patient, such as radial access, for insertion of a catheter 12 into the patient separate from the surgery to create the AV fistula 4. Thus, the embodiments described above may be utilized to ablate nerves in the target tissue 102 while the patient is under anesthesia for the creation of the AV fistula 4, thereby permanently anesthetizing the target tissue as part of creating the AV fistula 4.

Turning to FIGS. 1E-1F, in some embodiments, the anesthetizing system 2 is configured to be placed through at least one bodily vessel of a subject to a position at or near a target tissue. The anesthetizing system 2 is configured for placement of the ablation element 110 inside the body during use. The ablation element 110, when placed intravascularly, is most efficiently introduced to the site of the AV fistula 4 by radial access, and then moved down the radial artery in the arm to its intended placement for performing neuromodulation. While accessing the radial artery, and closing the access point, are significantly easier than accessing and closing the femoral artery, that process is still more complicated than inserting a needle into the AV fistula 4.

In some embodiments, the spacer 130 is a balloon that radially surrounds the ablation element 110. As discussed previously, the balloon may be a compliant, semi-complaint, or non-compliant medical balloon 14. In other embodiments, the spacer 130 may be a helical wire, expandable frame, or other structure. In such embodiments, blood in the radial artery or other artery may flow onto the ablation element 110, thereby cooling the ablation element 110 in use. In certain embodiments, the spacer 130 may be configured to position the ablation element 110 relative to one or more of at least a portion of the epidermis 104a or the dermis 104b.

In certain embodiments, the anesthetizing system 2 includes a catheter 10 or hub 16 configured to aid placement of the catheter 12 through at least one bodily vessel of a subject to a position at or near a target tissue. The catheter shaft 12 may be inserted into a blood vessel of a subject, and advanced through the vasculature of the subject until the ablation element 110 is in proximity to a target tissue. In some embodiments, the catheter shaft 12 is inserted into the radial artery of a subject, through a radial artery access site, in a conventional manner. Such an insertion of a catheter into a radial artery access site is described in, for example, US Pat. App. Pub. No. 2022/0378461, which is incorporated by reference herein in its entirety. Insertion of the catheter shaft 12 into the vasculature of the subject through a radial artery access site may be advantageous where the target tissue is located in the arm; the distance that the catheter shaft 12 travels through the vasculature to the target tissue is minimized, and a radial artery access site is easier to close at the end of the procedure than a femoral artery access site. In some embodiments, the catheter shaft 12 is inserted into the femoral artery of a subject, through a femoral artery access site, in a conventional manner. Such an insertion of a catheter into a femoral artery access site is described in, for example, U.S. Pat. No. 9,981,108, which is incorporated by reference herein in its entirety.

In certain embodiments, the catheter shaft 12 may be configured to be placed through at least one bodily vessel of a subject to a position at or near a target tissue without the aid of a catheter. In certain embodiments, the ablation element 110 may be placed over a device shaft, e.g., catheter shaft 12, and configured to emit ablation energy radially outward or laterally outward from the device shaft.

In certain embodiments, the ablation element 110 is a substantially cylindrical ultrasound transducer, and configured to emit ultrasound energy radially outward across its entire circumference. Such a cylindrical transducer is described in the following patents and published applications, the entire contents of each of which are incorporated by reference herein in their entirety: U.S. Pat. Nos. 6,635,054; 6,763,722; 7,540,846; 7,837,676; 9,707,034; 9,981,108; 10,350,440; 10,456,605; 10,499,937; US Pat. Pub. No. 2022/0378461; and PCT Publication No. WO 2012/112165. In such embodiments, the target tissue 102 is treated, as well as tissue circumferentially around the AV fistula 4. For some patients, treatment of a larger volume than the target tissue 102 may be desirable, particularly for patients who are sensitive to pain and/or who may have significant anxiety associated with dialysis. In other embodiments, the ablation element 110 is configured to emit ultrasound energy directionally, toward the target tissue 102, such that other tissue is not treated. In such embodiments, the ablation element 110 may be substantially flat, or shallowly curved in a concave manner, and orientable toward the target tissue 102. In such embodiments, the anesthetizing system 2 includes a driver (not pictured) configured to rotate or align at least one of the catheter shaft 12 and the ablation element 110 relative to the target tissue. Alternately, the clinician may simply move the catheter shaft 12 through the radial artery in the preferred orientation, and/or manipulate the catheter shaft 12 when the ablation element 110 has reached the AV fistula 4, in order to orient the ablation element 110 in a desired direction at the treatment site.

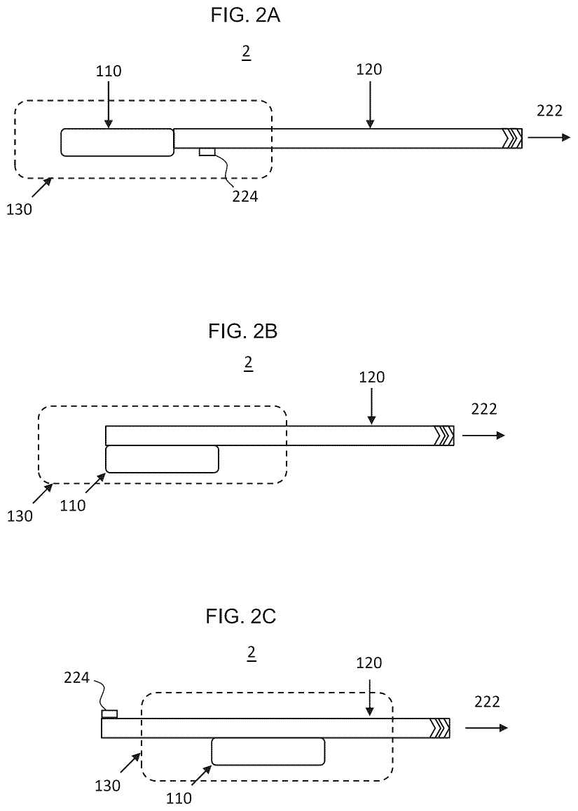

In certain embodiments, at least one of the catheter shaft 12 or the ablation element 110 may include a marker 224 configured for determining an orientation of the ablation element 110 relative to at least one of the target tissue 102 or the portion of skin (FIG. 2A). Examples of a marker include but are not limited to a radiopaque marker, a magnet, or a metallic fiducial. In an example, the marker 224 may be positioned and aligned with the ablation element 110 such that detection of the marker can infer or calculate the orientation of the ablation element 110 in three dimensions (3D). In an example, the marker 224 can work in coordination with a spatial sensor configured to determine one or more of a position or orientation of the ablation element 110, directly or indirectly by inference of known system geometry. In another example, the marker 224 may be visible to a clinician in a manner that allows the clinician to place the catheter shaft 12 and ablation element 110 in a desired position or orientation relative to the subject.

In some embodiments, the spacer 130 is an inflatable balloon coupled to the catheter shaft 12, where the inflatable balloon may be configured to inflate at or near the target tissue such that the inflated balloon at least partially contacts a bodily vessel wall. Fluid may be pumped into the balloon to inflate it. For example, the balloon may be inflated with a liquid such as saline or sterile water, or with a medically suitable gas. In some embodiments, that fluid is pumped through the balloon during operation of the ablation element 110, in order to cool the ablation element 110 and adjacent tissue while the ablation element 110 emits energy. Warmed fluid may be pumped out of the balloon, or may exit the balloon through weeping features. Further balloon and cooling designs are described in U.S. Patent Pub. No. US2022/0265302), which is incorporated herein by reference in its entirety.

The carrier 120 may include at least one electrical coupling for connecting the ablation element 110 to the electronics 22. In certain embodiments, at least one of the balloon 130 or the carrier 120 includes at least one fluid port 24 in fluid communication with a fluid source 28. In an aspect, the fluid source 28 includes a cooling agent. In an example, the cooling agent may be configured to circulate through the balloon 130 and to cool a portion of skin adjacent to the target tissue 102 during energy delivery.

In certain embodiments, the anesthetizing system 2 can be operatively connected (222) to, or include, a generator, e.g., electronics 22, configured to be in operative communication with the ablation element 110 and provide the ablation element 110 with energy that the ablation element 110 converts (and/or transmits) as neuromodulating energy into target tissue 102. The ablation energy may be sufficient to anesthetize or reduce a sensation of pain in some or all the target tissue without injuring another portion of the skin or other tissue.

In an example, the ablation element 110 may be a piezoelectric transducer. In an example, the ablation element 110 may be a cylindrical transducer 130 configured to deliver unfocused ultrasound energy 602c (FIG. 6C). In an example, the ablation element 110 may be a cylindrical piezoelectric transducer. In an example, unfocused energy emitted from a cylindrical piezoelectric transducer may be modified by a spacer 130 such as a balloon, as described above. In some embodiments, the spacer 130 may preferentially direct acoustic energy in certain directions over others. Alternatively or in combination, the shape of the transducer (for example, the cross-sectional shape of a cylindrical or elongate transducer) may be designed to preferentially direct acoustic energy in one or more directions over others. In another example, the ablation element 110 may be a unidirectional piezoelectric element and the orientation of the ablation element 110 may be used to modify the ablation energy. A unidirectional piezoelectric element may be flat, or shallowly curved. In some embodiments, a deflective covering 620 may be provided to one or more of modify, deflect, or redirect at least a portion of acoustic energy generated by the ablation element 110 (FIG. 6D).

Other embodiments of ablation element 110 include those using geometric focusing, as shown in FIGS. 6G-6J. As shown in FIGS. 6G and 6I, a ablation element 110 may be a single transducer element 600g shaped to focus the ablation energy generated (e.g., ultrasound energy) into a single focal point 630. As shown in FIGS. 6H and 6J, instead of a single transducer element, the ablation element 110 may comprise a plurality of transducer elements 600h arranged to focus the ablation energy generated (e.g., ultrasound energy) into a single focal point 630. Energy at or near the single focal point 630 may be sufficient to additionally ablate a volume of tissue 635 near the single focal point 630, as shown in FIGS. 6I-6J. Ablation element(s) 110 relying on geometric focusing may be operated at a frequency between 3 and 12 MHz or at a small total power, for example, 1 to 10 W. In some embodiments, focusing is performed across or through an area or volume of target tissue, and line focus, for example, may be performed with a cylindrical transducer element 110 (i.e., an elongate transducer element that is concave at one of its sides to focus the generated ablation energy) as shown in cross-sectional view in FIG. 6K, or weak focus or a pre-focal zone (FIGS. 6I, 6J, 6K) may be used. Generally, treatment depth can be adjusted or designed by adjusting one or more of frequency, geometry, time, or power.

Another category of ablation element 110 includes those using waveguide or lens focusing, as shown in FIGS. 6L-6M. These ablation elements 110 may be ultrasound transducers that can take any shape, forming focal points, lines, or other shapes. An example of a flat transducer 130 coupled to a lens 640, for example, made of a metal such as aluminum, is shown in FIG. 6L. An example of a flat transducer 130 coupled to an acoustic lens 645 is shown in FIG. 6M.

Another category of ablation elements 110 includes those using phased array transducers or transducer elements, such as a phased array ultrasound transducer. With phase adjustment, a variety of geometries suitable for treatments may be designed. In certain embodiments, the ablation element 110 may be at least one of an annular phased array transducer or a linear phased array transducer.

### Sensor

In certain embodiments, the anesthetizing system 2 includes at least one subject sensor configured to detect a parameter associated with the subject. Non-limiting examples of subject sensors include one or more of a temperature sensor, blood flow meter, skin conductance meter, heart-rate monitor, or muscle activity monitor. In certain embodiments, an exemplary system includes at least one environmental sensor configured to detect an amount of ablation energy emitted by the ablation element 110 in one or more directions. An example of an environmental sensor includes a thin-film array sensor 624 (FIG. 6F) configured to sense ablation energy.

In certain embodiments, one or more sensors can be positioned near the portion of skin, within a bodily vessel, or at or near the target tissue. In an example, a heart-rate monitor may include separate electrocardiogram (ECG) electrodes coupled with an alternating current (AC) amplifier and a muscle activity monitor may include separate EMG electrodes coupled with the AC amplifier.

In certain embodiments, ablation energy delivered from the ablation element 110 may be modified based on a sensor reading from either or both of a subject sensor and an environmental sensor. For example, at least one parameter of the ablation energy can be adjusted by the electronics 22 in response to feedback information received from a temperature sensor, a blood flow meter, a skin conductance meter, a heart rate monitor, a muscle activity monitor, and/or a thin-film array sensor 624 (FIG. 6F). In certain embodiments, at least one parameter of the ablation energy can be adjusted by a user of the system based on information collected from at least one sensor 624.

Optionally, some embodiments of the exemplary anesthetizing system 2 include a spatial sensor configured to determine one or more of a position or orientation of the ablation element 110. In an example, the spatial sensor can work in coordination with a marker 224. Examples of spatial sensors can include inertial measurement unit (IMU) devices and z-axis magnetometers.

In certain embodiments, information related to the ablation energy, the patient, including one or more properties of the skin/tissue (e.g., electrical, thermal, etc.), the electrodes, and the cooling mechanism can also be provided via a user interface and used to adjust at least one parameter of the ablation energy or cooling mechanism.

### Methods

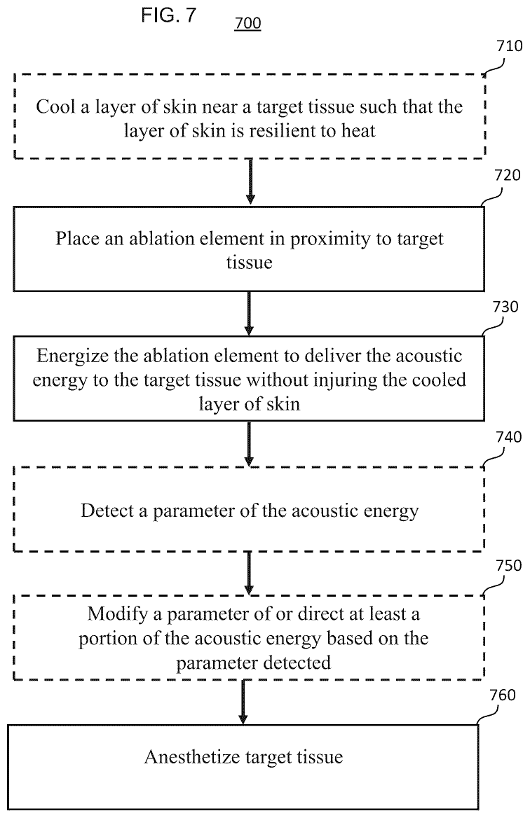

Turning to FIG. 7, an exemplary method 700 for anesthetizing target tissue at or adjacent a portion of skin utilizing an exemplary anesthetizing system 2 extracorporeally is described. The method 700 may be advantageously performed in conjunction with surgery to create the AV fistula 4. During that surgery, depending on the patient and the choice of the clinician, the patient is placed under general anesthesia, regional anesthesia, or local anesthesia. In all of those cases of surgical anesthesia, the patient would not experience pain when neuromodulation was applied to target tissue 102 during the surgery performed to create the AV fistula 4. Further, the extracorporcal administration of ablation energy from the ablation element 110 may assist in the anesthesia of the target tissue during the surgery to create the AV fistula 4. Such extracorporcal administration of ablation energy also does not require a separate entry into the body of the patient, such as radial access, for insertion of a catheter 12 into the patient separate from the surgery to create the AV fistula 4. Thus, the embodiments described above may be utilized to ablate nerves in the target tissue 102 while the patient is under anesthesia for the creation of the AV fistula 4, thereby permanently anesthetizing the target tissue as part of creating the AV fistula 4.

In some embodiments, a subject may have an AV graft instead of an AV fistula 4 for dialysis. An AV graft is typically located under the skin, and the method 700 may be suitable for subjects with AV grafts, where the graft is made of a non-acoustically transparent material that may block acoustic emission or other emission of acoustic energy or may have a marker to facilitate the determination of one or more of the depth, range, or location of the AV graft. The method 700 is performed in the same manner described below whether the method is performed in conjunction with an AV fistula 4 or an AV graft, or whether the method is performed in conjunction with creating an AV fistula 4 or implanting an AV graft.

Optionally, at block 710, a layer of skin near above the target tissue 102 may be cooled, such as with an ice pack 140, such that the layer of skin may be partially resilient to heat. Also optionally at block 710, the patient may be given a local or regional anesthetic at the treatment site, or the patient may be placed under general anesthesia, depending on factors such as the professional judgment of the clinician and whether other procedures will be performed on the patient during treatment. In certain embodiments, the block 710 of cooling a layer of skin near a target tissue such that the layer of skin may be partially resilient to heat can be done in several ways. For example, using an anesthetizing system 2 such as that of FIG. 1C, which is placed extracorporcally, the cooling can be applied on the surface of the epidermis 104a, such that the method 700 includes extracorporcally cooling a layer of skin near the target tissue 102; as a result, that layer of skin may beat least partially resilient to heat generated from the application of acoustic energy to target tissue 102. In an aspect, the layer of skin being cooled may be the epidermis 104a, the dermis 104b, and/or subcutaneous tissue 104c. In an example, cooling may be provided by placing a cooling source on or near a portion of skin in proximity to the target tissue 102. In an example, the cooling source may be an ice pack 140 (FIG. 1F). In certain embodiments, where the acoustic energy is unfocused ultrasound, cooling may be needed to protect the skin from excessive heat. For example, cooling of the skin may be provided for 5 seconds to 30 seconds.

As another example, using an anesthetizing system 2 such as that of FIG. 1E, cooling can be applied intravascularly. In such an example, as described above, the spacer 130 may be a balloon surrounding at least a portion of the ablation element 110. Cooling fluid may be delivered to the balloon prior to the ablation energy being delivered, concurrently with the ablation energy being delivered, or after the ablation energy is delivered.

As another example, the patient may place their arm, where the AV fistula 4 is to be created in or exists in the arm, into a container of water or acoustic gel. Water is a suitable transducing medium for ultrasound energy. The use of water both cools the target tissue 102 during actuation of an ultrasonic transducer used as a ablation element 110, and acoustically couples the ultrasonic transducer to the surface of the epidermis 104a in an effective manner. The water or acoustic gel may be cooled prior to, or while, the patient's arm is in the container, to cool the target tissue 102 more effectively.

Next, at block 720, a ablation element 110 is placed in proximity to the skin of the patient, above the target tissue 102. The spacer 130 associated with the ablation element 110 may be placed in contact with the skin of the patient, as described above. Also as described above, a transducing medium may be placed within or in association with the spacer 130, where the ablation element 110 is an ultrasound transducer, thereby facilitating acoustic coupling between the ablation element 110 and the skin of the patient. Where the ablation element 110 includes RF electrodes, microwave emitters, or one or more emitters of energy other than ultrasound, the ablation element 110 may be placed in direct contact with the skin of the patient, without the use of the spacer 130.

Next, at block 730, the ablation element 110 is energized by the electronics 22 to emit energy into the target tissue 102. Where the ablation element 110 is an ultrasound transducer, that transducer is energized to emit unfocused ultrasound into the target tissue 102. The distance between the ablation element 110 and the target tissue 102 is set such that the unfocused ultrasound will transfer most of its energy to the target tissue 102 rather than surrounding tissue. In other embodiments, at block 730, the transducer is energized to emit focused ultrasound into the target tissue 102. In other embodiments, the ablation element 110 is energized to emit RF energy, microwave energy, electricity, or other form or forms of energy into the target tissue 102.

Optionally, where the ablation element 110 is an ultrasound transducer, the method may include a block 740 of detecting a parameter of the acoustic energy emitted by the ablation element 110. The block 740 of detecting a parameter of the acoustic energy can be done several ways. For example, a parameter can be detected using a subject sensor, environmental sensor, or spatial sensor as described above.

Optionally, where the ablation element 110 is an ultrasound transducer, the method may include a block 750 of modifying a parameter of the acoustic energy or directing at least a portion of the acoustic energy, based on the detecting performed at block 740. In certain embodiments, block 750 may include modifying the acoustic energy based on the sensed acoustic energy.

At block 760, the application of energy to the target tissue 102 from the ablation element 110 ceases when the target tissue 102 has been anesthetized. Where the ablation element 110 emits acoustic energy, the duration of application of acoustic energy may depend on the depth of the target tissue 102, the volume of the target tissue 102, and the characteristics of the emitted acoustic energy. For example, the acoustic energy in some embodiments may comprise focused ultrasound and the focused ultrasound may be delivered for less than one second to, e.g., ablate target tissue.

Depending on the length of the ablation element 110 relative to the length of the AV fistula 4, after performing the method 700, the ablation element 110 is moved relative to the target tissue 102, and the method 700 is repeated to treat a different portion of the target tissue 102. The method 700 can be repeated one or more times, until the entirety of the target tissue 102 has been treated. Treatment of the entirety of the target tissue 102 does not require geometrical precision relative to the volume of the target tissue 102; rather, it means that ablation energy has been delivered into the target tissue 102 at locations that are sufficient to ablate the nerves in the target tissue 102. Where the target tissue 102 is divided into two spaced-apart volumes, the method 700 is performed on each of those spaced-apart volumes at least once.

In some embodiments, the ablation element 110 is a focused ultrasound (HIFU) transducer. That transducer may be concave or otherwise shaped to focus ultrasound from the ablation element 110 into the target tissue 102. In some embodiments, the HIFU transducer may be a cylinder or half-cylinder, where the rear portion thereof is covered with an ultrasound reflector, nonacoustically transmissive insulation, or backing material that is nonacoustically transmissive. The HIFU transducer may be focused to a line, an area, or other shape. Where the HIFU transducer is focused to a line, that transducer is oriented such that the line is oriented generally along the width of the forearm, and the method 700 is repeated several times as the HIFU transducer is moved down the length of the target tissue 102. In this way, the line focus of the HIFU transducer ablates the nerves in the target tissue 102 at one or more different locations along the target tissue 102. As an example, the HIFU transducer is actuated for 0.5-15 seconds each time, at 7-20 MHZ, at a power from 10-100 W based on the vessel size and the size of the focal region (e.g., 1×0.5 cm), while the vicinity of treatment is cooled with 0.1-50 ml/minute of room temperature water, or while the patient's arm or other tissue is surrounded by water at substantially 5° C.

An exemplary method for anesthetizing target tissue at or adjacent a portion of skin utilizing an exemplary anesthetizing system includes placing a ablation element 110 over a portion of skin of a subject, the portion of skin being positioned at or near a target tissue 102 and the ablation element 110 being in communication with a electronics 22 configured to generate energy, cooling at least one of a portion of the target tissue 102 or a portion of skin at or near the target tissue resulting in greater resiliency to heat generated from the energy, and energizing the ablation element 110 to deliver the energy to the target tissue 102, the energy being sufficient to anesthetize a portion of the target tissue 102 without injuring the portion of the skin and reduce the sensation of pain experienced by a subject at a needle insertion site in proximity to the AV fistula 4.

In certain embodiments, the method 700 includes directing at least a portion of the energy toward the portion of skin or the target tissue 102. In an example, at least a portion of the energy may be directed by a spacer 130 associated with the ablation element 110.

In certain embodiments, the method 700 includes determining at least one of a depth, a thickness, or a volume of the target tissue 102.

In certain embodiments, the method 700 includes determining an orientation of the ablation element 110. In one example, determining an orientation of the ablation element 110 includes energizing the ablation element 110 to deliver non-ablative energy and detecting the non-ablative energy. In an example, determining an orientation of the ablation element 110 includes detecting a marker 224 aligned with the ablation element 110. In an example, the marker 224 comprises at least one of a radiopaque marker, a magnet, an echogenic marker, or a metallic fiducial.

In certain embodiments, the method 700 includes determining an orientation of a unidirectional ablation element 110. In an example, the determining an orientation of the ablation element 110 includes detecting a marker 224 (described above) aligned with the ablation element 110. In certain embodiments, determining an orientation of the ablation element 110 includes energizing the ablation element 110 to deliver non-ablative energy and detecting the non-ablative energy.

In certain embodiments, the method 700 includes detecting a parameter of the acoustic energy using a sensor 624 (subject sensor or environmental sensor) and modifying the parameter of the energy emitted from the ablation element 110 based on the acoustic energy sensed by the sensor 624.

In certain embodiments, the method 700 includes sending non-ablative energy to image the target tissue 102 or non-target tissue. In an example, the non-ablative energy is delivered by the ablation element 110. In an example, the non-ablative energy is delivered by an imaging or guidance system (not pictured) for aiding placement of the catheter system 10.

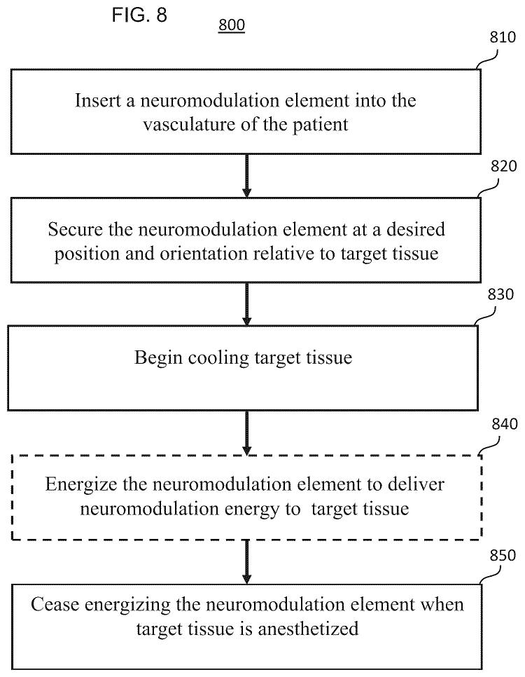

Turning to FIG. 8, an exemplary method 800 for anesthetizing target tissue at or adjacent a portion of skin in proximity to target tissue 102, utilizing an exemplary anesthetizing system 2 intravascularly, is described. The method 700 may be suitable for subjects currently having a functional AV fistula 4. In some embodiments, a subject may have an existing AV fistula 4 but the cannulation of the AV fistula, for example, during dialysis, may be painful and the method 800 may be performed to ameliorate cannulation pain. The intravascular administration of ablation energy from the ablation element 110 is administered minimally invasively (i.e., interventionally) through the vasculature, thereby avoiding the need for surgery. Further, surgery in proximity to the AV fistula 4 is undesirable, due to the high rate of blood flow therethrough. Even a minor error in such surgery, which results in even a small incision in the AV fistula 4, may result in significant blood loss, making such surgery risky. Thus, the use of a catheter 12 and an interventional procedure is safer for the patient, and the puncture site for radial access or other access heals more easily and quickly than an incision or incisions that would be made during surgery.

In some embodiments, a subject may have an existing AV fistula 4 and the method 800 may be performed during a procedure to modify the AV fistula 4. In some embodiments, a subject may have an AV graft instead of an AV fistula for dialysis. The AV graft is typically located under the skin, and the method 800 may be suitable for subjects with AV grafts; the graft may be made of a non-acoustically transparent material that may block acoustic emission or other emission of acoustic energy or may have a marker to facilitate the determination of one or more of the depth, range, or location of the AV graft. The method 800 is performed in the same manner described below whether the method is performed in conjunction with an AV fistula 4 or an AV graft, or whether the method is performed in conjunction with creating an AV fistula 4 or implanting an AV graft.

At block 810, the ablation element 110 is inserted into the vasculature of the patient and advanced into proximity to the target tissue 102 via a catheter shaft 12. As described above, advantageously the ablation element 110 is placed into a radial artery and advanced a short distance along the radial artery into proximity to the target tissue 102. A clinician makes an incision or puncture at an access site, and inserts the distal end of the catheter shaft 12 into the vasculature of the subject through the access site. As described above, the access site may be made in the femoral artery or radial artery, or in any other suitable access location in the vasculature of the subject; the method 800 is not limited to femoral and/or radial access to the vasculature. After the distal end of the catheter shaft 12 and the associated ablation element 110 have been inserted into the vasculature of the subject, the catheter shaft 12 is advanced through the vasculature of the subject until the ablation element 110 is located substantially in proximity to the target tissue 102. Contrast agents and angiography are not required to position the ablation element 110 at block 810, due to the relative proximity of the ablation element 110 to the surface of the epidermis 104a. A clinician may observe a slight bulge in the epidermis 104a due to motion of the ablation element 110, or may palpate the epidermis 104a to feel the location of the ablation element 110 within the AV fistula 4. Contrast agent is toxic, and angiography utilizes ionizing radiation, and the elimination of both enhances safety for both the patient and the clinician. However, contrast agent and/or angiography may be performed at the discretion of the clinician.

At block 820, the ablation element 110 is secured at a desired position and orientation relative to the target tissue 102. As described above, the spacer 130 may be a balloon when the anesthetizing system 2 is utilized intravascularly. Where the spacer 130 is a balloon, at block 820 the balloon is inflated in a manner as described above, and the walls of the balloon contact the wall of the blood vessel in which the neuromodulation assembly 110 is placed. Contact between the balloon and the wall of the blood vessel secures the balloon, and thus the ablation element 110, in place relative to the blood vessel and relative to the target tissue 102. In certain embodiments, at block 820, an orientation of a unidirectional ablation element 110 is determined prior to securing it relative to the target tissue 102. As one example, determining an orientation of the ablation element 110 includes detecting a marker 224 (described above) aligned with the ablation element 110. In certain embodiments, determining an orientation of the ablation element 110 includes energizing the ablation element 110 to deliver non-treatment energy and detecting the non-treatment energy such as by a sensor or sensors 624.

At block 830, cooling the target tissue may begin. Block 830 may be performed before, during, and/or after block 840. As described above, fluid used to inflate the balloon may also be utilized to cool the balloon, and thus tissue in contact with the balloon as well, such as tissue of a blood vessel wall, and other tissue adjacent thereto. Cooling fluid may be circulated through the balloon and out of the patient, or may enter the bloodstream via weeping features defined in the balloon. Due to the closeness of the target tissue 102 to the surface of the skin, cooling instead, or in part, may be performed from the exterior of the body of the subject. As one example, cooled gel packs, ice, cold water or other fluid, or the like may be applied to the skin of the patient in proximity to the target tissue 102 for cooling. Such cooling items may be placed near the target tissue 102 before, during, and/or after energy is emitted from the ablation element 110. The cooling fluid cools the wall of the AV fistula 4, while allowing the target tissue 102 between the blood vessel and the surface of the epidermis 104a to reach the desired target temperature. In some embodiments, the flow rate of the cooling fluid through the balloon embodiment of the spacer 130 may be 0.1-60 ml/minute. In other examples, that flow rate may be 0.1-50 ml/minute, 0.1-40 ml/minute, 0.1-30 ml/minute, 0.1-20 ml/minute, 0.1-10 ml/minute, 0.1-5 ml/minute, 0.1-4 ml/minute, 0.1-3 ml/minute, 0.1-2 ml/minute, 1-60 ml/minute, 1-50 ml/minute, 1-40 ml/minute, 1-30 ml/minute, 1-20 ml/minute, 1-10 ml/minute, 1-5 ml/minute, 1-4 ml/minute, 1-3 ml/minute, 1-2 ml/minute, 2-60 ml/minute, 2-50 ml/minute, 2-40 ml/minute, 2-30 ml/minute, 2-20 ml/minute, 2-10 ml/minute, 2-5 ml/minute, 2-4 ml/minute, 2-3 ml/minute, 5-60 ml/minute, 5-50 ml/minute, 5-40 ml/minute, 5-30 ml/minute, 5-20 ml/minute, 5-10 ml/minute, 5-9 ml/minute, 5-8 ml/minute, 5-7 ml/minute, 5-6 ml/minute, 10-60 ml/minute, 10-50 ml/minute, 10-40 ml/minute, 10-30 ml/minute, 10-20 ml/minute, 20-60 ml/minute, 20-50 ml/minute, 20-40 ml/minute, 20-30 ml/minute, 30-60 ml/minute, 30-50 ml/minute, 30-40 ml/minute, 40-60 ml/minute, or 40-50 ml/minute. In some examples, the flow rate of the cooling fluid is substantially 40 ml/minute. As one example, where the ablation element 110 is an ultrasound transducer, the electronics 22 may provide power to the transducer at 25-40 W, for 7 seconds, while the cooling fluid is circulated through the balloon at a flow rate of substantially 40 ml/minute.

In some embodiments, the spacer 130 may be an expandable cage or network of struts, which allows blood to flow through the spacer 130 into contact with the ablation element 110; in other embodiments, the spacer 130 may be omitted, such that the ablation element 110 is positioned in the AV fistula 4 with nothing between the ablation element 110 and the wall of the AV fistula 4. In such embodiments, cooling fluid is not required, because the ablation element 110 may be cooled sufficiently by the high flow of blood within the AV fistula 4 for the short duration of actuation of the ablation element 110. Embodiments that do not require cooling fluid may be less complex and less costly than embodiments that require cooling fluid and the mechanism(s) for its delivery.

At block 840, the ablation element 110 is energized to deliver ablation energy to the target tissue 102 based on at least one of the orientation of the ablation element 110, the depth, the thickness, and the volume of the target tissue 102 without substantially injuring the cooled layer of skin and non-target tissue. Where the ablation element 110 is an ultrasound transducer, the transducer may be energized to emit unfocused ultrasound circumferentially around the blood vessel in which the transducer is located. In other examples, the transducer may be unidirectional, and that transducer is energized to emit unfocused ultrasound in a direction toward the target tissue 102. In other examples, the transducer may be configured to emit focused ultrasound, and that transducer is energized to emit focused ultrasound toward the target tissue 102.

At block 850, the application of energy to the target tissue 102 from the ablation element 110 ceases when the target tissue 102 has been anesthetized.

Although the above blocks show methods 700, 800 of treating a patient in accordance with embodiments, a person of ordinary skill in the art will recognize many variations based on the teaching described herein. The blocks may be completed in a different order. Blocks may be added or deleted. Some of the blocks may comprise sub-blocks. Many of the blocks may be repeated as often as beneficial to the treatment. For example, the method 700, 800 may be considered incomplete and repeated at least partially if the subject is still capable of feeling pain at the target tissue, such as indicated by a needle poke.

One or more of the blocks of the methods 700, 800 may be performed with the electronics 22 described herein, for example, one or more of the processor or logic circuitry, such as programmable array logic for a field programmable gate array. The electronics 22 may be programmed to provide one or more of the blocks of the methods 700, 800, and the program may comprise program instructions stored on a computer readable memory or programmed blocks of the logic circuitry such as the programmable array logic or the field programmable gate array, for example.

Both the extracorporeal and the intravascular methods of treatment described above are intended to neuromodulate the nerve or nerves in the target tissue 102 to permanently anesthetize them. As described above, the acoustic dose or the amount of other ablation energy delivered to the target tissue 102 is selected to cause such permanent anesthesia. Different patients have different anatomy and different biochemistry, so for some patients, a particular acoustic dose or particular amount of other ablation energy may cause anesthesia of the target volume for a duration, after which feeling returns to the target tissue 102. Such duration is measured in months or years. If feeling does return to the target tissue 102, which is expected to be a rare event, a clinician and patient may decide to perform one of the methods 700, 800 on the patient a second time, after which permanent anesthesia is expected to be achieved. It is possible that a patient will experience phantom pain after the target tissue 102 is anesthetized, and such phantom pain can be treated in a same or similar manner as other phantom paid that may be experienced by a patient.

In other embodiments of the present disclosure, neuromodulation and permanent anesthesia of the target tissue 102 may be achieved without the system described above, by the use of neurotoxic chemicals. In such embodiments, a solution of one or more neurotoxic chemicals may be injected directly into the target tissue 102 to neuromodulate the nerves therein without the need for cooling. Such a solution may include, but is not limited to, ethanol, acetic acid, ethylene glycol, and/or isopropanol, individually or as a combination of two or more. In other embodiments, cryogenic liquid and/or gas may be applied to the target tissue 102 to neuromodulate the nerves therein such as by injecting that fluid into the target tissue 102, or a device at cryogenic temperature may be applied to the surface of the epidermis 104a above the target tissue 102. Other modalities instead, or additionally, may be used to neuromodulate the nerves in the target tissue.

In other embodiments of the present disclosure, a needle transducer may be used as a ablation element 110. The needle transducer may be inserted into the target tissue 102 and actuated to emit ultrasound energy into the target tissue 102.

While embodiments of the present disclosure have been shown and described herein, it will be obvious to those skilled in the art that such embodiments are provided by way of example only. It is not intended that the present disclosure be limited by the specific examples provided within the specification. While the present disclosure has been described in conjunction with an AV fistula 4 for use in dialysis, it will be apparent to those skilled in the art that the present disclosure may be utilized in conjunction with AV fistulas 4 created for other purposes, and in conjunction with AV fistulas 4 located anywhere in the body. Further, it will be apparent to those skilled in the art that the present disclosure may be used to anesthetize and/or ablate nerves in proximity to blood vessels, even if an AV fistula 4 is not present in those blood vessels. While the present disclosure has been described with reference to the specification, the descriptions and illustrations of the embodiments herein are not meant to be construed in a limiting sense. Numerous variations, changes, and substitutions will now occur to those skilled in the art without departing from the scope of the present disclosure. Furthermore, it shall be understood that all aspects of the present disclosure are not limited to the specific depictions, configurations or relative proportions set forth herein which depend upon a variety of conditions and variables. Various alternatives to the embodiments of the present disclosure described herein may be employed in practicing the present disclosure. It is therefore contemplated that the present disclosure shall also cover any such alternatives, modifications, variations, or equivalents. Those and others are within the scope of the following claims.

What is claimed is:

1. A system for treating at least one nerve in target tissue in proximity to an AV fistula of a patient having an epidermis, comprising:
a ablation element configured to emit ablation energy to the at least one nerve in proximity to an AV fistula;
a generator electrically coupled to the transducer, wherein the generator transmits electrical energy to the transducer; and
a spacer connected to the ablation element, wherein at least part of the spacer is configured to contact the surface of the epidermis in proximity to the nerves surrounding the AV fistula, and thereby space the ablation element a particular distance from the surface of the epidermis;
wherein the ablation energy is sufficient to anesthetize at least one of the nerves in the target tissue.

2. The system of claim 1, wherein the spacer contacts the epidermis to maintain a substantially fixed distance between the transducer and the epidermis.

3. The system of claim 1, wherein the spacer contacts the epidermis to maintain an adjustable distance between the transducer and the epidermis.

4. The system of claim 1, wherein the ablation element is configured for intravascular insertion and operation; wherein the spacer is a balloon that radially surrounds the ablation element.

5. The system of claim 4, wherein the balloon is configured for circulation of a cooling fluid therethrough.

6. The system of claim 4, further comprising
a marker adjacent the ablation element; and
a spatial sensor connected to the generator, wherein the spatial sensor is configured to sense the marker and thereby determine one or more of a position or orientation of the ablation element.

7. The system of claim 1, wherein the ablation element is configured for intravascular insertion and operation; and wherein the spacer is selected from the group consisting of: an inflatable balloon coupled to the catheter, and a helical wire.

8. The system of claim 1, wherein the ablation element is an ultrasound transducer, and the ablation energy emitted therefrom is ultrasound energy.

9. The system of claim 8, wherein the ultrasound transducer is substantially cylindrical, and emits unfocused ultrasound energy radially outward circumferentially therefrom.

10. The system of claim 8, wherein the ultrasound transducer is a unidirectional ultrasound transducer, and emits unfocused ultrasound energy substantially unidirectionally therefrom.

11. The system of claim 8, wherein the ultrasound transducer is substantially flat.

12. The system of claim 8, wherein the ultrasound transducer is configured to emit focused ultrasound.

13. The system of claim 8, further comprising a transducing medium held by the spacer, located between the transducer and the epidermis.

14. The system of claim 1, further comprising at least one sensor coupled to the generator, wherein the at least one sensor is configured to detect at least one of: an amount of ablation energy emitted by the transducer, temperature, blood flow, skin conductance, heart rate, and muscle activity.

15. A method for treating at least one nerve in target tissue in proximity to an AV fistula of a patient, comprising:
placing an ablation element associated with a spacer in proximity to the target tissue;
cooling tissue in proximity to the at least one nerve; and
energizing the ablation element to deliver ablation energy to the target tissue until at least one nerve in the target tissue is anesthetized.

16. The method of claim 15, wherein the at least one nerve is a dermal nociceptor.

17. The method of claim 15, wherein the placing is extracorporeal.

18. The method of claim 17, wherein the placing comprises placing the spacer in contact with the epidermis near nerves surrounding an AV fistula, and wherein the cooling comprises providing one of a liquid and a gel within the spacer in contact with the epidermis.

19. The method of claim 17, wherein the spacer is transducing gel.

20. The method of claim 17, further comprising performing said energizing before creation of the AV fistula.

21. The method of claim 17, further comprising performing said energizing during creation of the AV fistula

22. The method of claim 15, wherein the placing is intravascular.

23. The method of claim 22, wherein the ablation element is connected to a catheter, and wherein the placing further comprises inserting the ablation element into a radial artery through a radial access and advancing the ablation element along the radial artery into proximity to the target tissue.

24. The method of claim 22, wherein the spacer is a balloon surrounding at least part of the ablation element; further comprising inflating the balloon after the placing; wherein the cooling comprises circulating liquid through the balloon.

25. The method of claim 22, wherein the spacer allows blood to contact the ablation element.

26. The method of claim 15, further comprising:
sensing a parameter of the ablation energy; and
modifying a parameter of the ablation energy based on the sensing.
